(19) United States
(12) Patent Application Publication
(10) Pub. No.: US 2012/0213706 A1
(43) Pub. Date: Aug. 23, 2012

(54) RECONSTITUTED TUMOR MICROENVIRONMENT FOR ANTICANCER DRUG DEVELOPMENT

(75) Inventors: Debabrata Banerjee, Bellerose, NY (US); Pravin J. Mishra, Bethesda, MD (US); Prasun J. Mishra, Gaithersburg, MD (US); Hui Gao, Walpole, MA (US); John Glod, North Brunswick, NJ (US)

(73) Assignee: UNIVERSITY OF MEDICINE AND DENTISTRY OF NEW JERSEY, Somerset, NJ (US)

(21) Appl. No.: 13/264,348

(22) PCT Filed: Nov. 12, 2009
(86) PCT No.: PCT/US09/64143

§ 371 Date: May 4, 2012

Related U.S. Application Data
(60) Provisional application No. 61/212,794, filed on Apr. 15, 2009.

(51) Int. Cl. C12Q 1/66; G01N 21/64; A61K 49/00; C12Q 1/02

(52) U.S. Cl. 424/9.2; 435/29; 435/8

(57) ABSTRACT

Extracellular matrix bioscaffolds capable of supporting the formation and growth of tumors from tumor cells introduced thereto containing tumor associated macrophages and carcinoma-associated fibroblast-like cells cultured under conditions effective to provide a cellular matrix capable of supporting the formation and growth of tumors from tumor cells introduced to the matrix. Bioscaffold kits and methods for using the bioscaffolds for testing, identifying and development of known or novel anti-cancer therapeutics are also disclosed.

### CROSS-REFERENCE TO RELATED APPLICATION

The instant application claims 35 U.S.C. §119(e) priority to U.S. Provisional Patent Application Ser. No. 61/212,794 filed Apr. 15, 2009, the disclosure of which is incorporated herein by reference.

### FIELD OF THE INVENTION

The present invention relates, generally, to an extracellular matrix bioscaffold that includes carcinoma-associated fibroblast-like cells and tumor associated macrophages supporting tumor cell growth and tumor formation, which may be used for the testing, identification and development of known or novel anticancer therapeutics.

### BACKGROUND OF THE INVENTION

Despite numerous advances and continuing research efforts, cancer is still one of the leading causes of human death worldwide. Understanding the environment surrounding cancer cell growth and motility is one avenue being explored and could provide useful information for the development of novel therapeutics. Current research suggests that cancer cell growth and invasion may be driven, at least in part, by the interactions between cells and a specific extracellular matrix (ECM). In the case of many epithelial-derived carcinomas, for example, a specialized ECM or basement membrane surrounds the primary tumor and is required for cell growth. With this in mind, many researchers have attempted to develop reconstituted basement membrane matrices that support cell growth in order to study the milieu of such carcinoma cells, as well as identify potential treatment target sites.

One such reconstituted basement membrane is set forth in U.S. Pat. Nos. 4,829,000 and 5,158,874. The membrane, or “matrigel,” set forth in these patents is rich in the extracellular matrix proteins laminin, collagen IV, heparan sulfate proteoglycans, entactin, and nidogen. It is formed by first extracting these components from Engelbreth Holm-Swarm (EHS) mouse sarcomas, then heating and polymerized the extract to form a three dimensional matrix. Such a matrix is embodied within the biological membrane and cell culture reagent BD Matrigel™ available from BD Biosciences. Indeed, this product is widely used by researchers for studying carcinoma cell interaction and identifying putative chemotherapeutic agents.

Many current methods for novel therapeutic evaluation rely on the detection of a change in carcinoma cell growth and motility within or through such a matrix. In particular, methods of assaying the effects of such therapeutics involve measuring a decrease in the number of cells (or the absence of cells) that grow within or penetrate through the matrix upon application of the agent. The use of such models, however, have certain drawbacks. In a large majority of instances, for example, in vitro results using such biological membranes do not correlate well with follow-up tests in vivo. Some researchers surmise that this may be because the murine-based Matrigel™ does not adequately mimic the actual cancer cell microenvironment. Thus, the myriad of cell types, growth factors, chemokines, and other relevant proteins and communication molecules that are involved in tumor cell growth and invasion are almost entirely ignored in the artificial environment. Such a drastic change in the tumor mileu from in vitro to in vivo test conditions could be a major reason why there is such a high incidence of drug failure. Accordingly, there remains a need in the art for more accurately replicating the tumor milieu or microenvironment, both in vitro and in vivo, which would lead to improved testing methodologies for chemotherapeutic compounds.

Beyond just drug screening assays, however, the increased understanding in the genetic variability of cancer cells makes specific and targeted treatment of a particular carcinoma genotype and/or phenotype increasingly possible and more desirable. Again, one current limitation in doing so is the inability to culture cells within an environment that substantially mimics the microenvironment within the patient. To this end, a matrix is also desirable that would replicate such conditions outside of the patient for the purpose of testing and identifying the effects of one or more known or novel agents on those cells. Such a matrix would, in certain instances, be adaptable to current in vitro or in vivo testing methodologies and would ultimately contribute to a personalized therapeutic strategy in treating carcinoma growth and invasion.

### SUMMARY OF THE INVENTION

The instant invention through its embodiments and examples addresses these needs.

In one embodiment, the instant invention provides an extracellular matrix bioscaffold in which tumor associated macrophages and carcinoma-associated fibroblast-like cells are cultured under conditions effective to provide a cellular matrix capable of supporting the formation and growth of tumors from tumor cells introduced to the matrix.

Carcinoma-associated fibroblast-like cells may include mesenchymal stem cells that are differentiated on a tumor conditioned medium. In a non-limiting embodiment, mesenchymal stem cells are differentiated on tumor conditioned medium for about 1 to 30 days. Resulting cells express stromal-derived factor-1 or may otherwise be positive for one or more biological markers selected from a-smooth muscle actin, vimentin, and fibroblast surface protein.

Tumor associated macrophages of the instant invention refer to a population of leukocytes exhibiting a macrophage phenotype that promotes tumor cell proliferation, metastasis and/or angiogenesis, or otherwise promotes chemotaxis of MSCs. In one non-limiting embodiment the tumor associated macrophages are phorbol ester differentiated leukocytes, such as HL-60 cells or U937 cells. In further embodiments, the HL-60 cells or U937 cells are differentiated in the presence of 3 nM of TPA for 96 hours.

Tumors capable of being grown in the extracellular matrix of the invention include any tumor cell line provided herein or otherwise known, which may be formed by incubating the tumor cells on the matrix for about one to about four days. In one embodiment, the tumor cells include one or more biological reporter genes. Such biological reporter genes may encode a reporter selected from a green fluorescence protein, luciferase, and combinations thereof. To this end, the biological reporter genes may be provided on one or more expression vectors that are transfected or otherwise expressed within the tumor cells.

In a further embodiment of the instant invention, methods for assaying the efficacy of a chemotherapeutic compound against a tumor cell line are provided, wherein the compound is administered to an extracellular matrix bioscaffold according to the present invention within which tumors are grown from the tumor cell line, and the size of the tumors are measured after sufficient time has elapsed for the compound to shrink the tumors. Reduction in tumor size may be determined by comparing a first value derived from the biological reporter before administration of the compound with a second value derived from the biological reporter after administration of the compound.

In a broader sense, methods are provided that assay the efficacy of tumor treatment methods against tumor cell lines. An extracellular matrix according to the present invention is provided within which tumors are grown that are suitable for testing, the treatment method is performed on the tumors in the extracellular matrix, and the size of the tumors is measured after sufficient time has elapsed for the treatment method to shrink the tumors.

Again, carcinoma-associated fibroblast-like cells may include mesenchymal stem cells that are differentiated on a tumor conditioned medium. In a non-limiting embodiment, mesenchymal stem cells are differentiated on tumor conditioned medium for about 1 to 30 days. Such cells may express stromal-derived factor-1 or may otherwise be positive for one or more biological markers selected from a-smooth muscle actin, vimentin, and fibroblast surface protein.

Tumor associated macrophages of the instant invention refer to a population of leukocytes exhibiting a macrophage phenotype that promotes tumor cell proliferation, metastasis and/or angiogenesis, or otherwise promotes chemotaxis of MSCs. In one non-limiting embodiment the tumor associated macrophages are phorbol ester differentiated leukocytes, such as HL-60 cells or U937 cells. In further embodiments, the HL-60 cells or U937 cells are differentiated in the presence of 3 nM of TPA for 96 hours.

Tumors capable of being grown in the extracellular matrix of the invention include any tumor cell line provided herein or otherwise known, which are incubated on the matrix for about one to about four days. In one embodiment the tumor cells include one or more biological reporter genes. Such biological reporter genes may encode a reporter selected from a green fluorescence protein, luciferase, and combinations thereof. To this end, the biological reporter genes may be provided on one or more expression vectors that are transfected or otherwise expressed within the tumor cells.

In addition to the foregoing, the instant invention further relates to methods for artificially establishing a tumor microenvironment for testing the effects of one or more chemotherapeutic agents against a cancer cell line in such an environment. In one embodiment, a method for providing a tumor microenvironment for testing one or more compounds for treating cancer is provided in which tumor cells are incubated in the extracellular matrix bioscaffold of the instant invention. The tumor cells may be incubated from one to four days in an extracellular matrix bioscaffold assembled in vitro. Alternatively, the tumor cells may be similarly incubated in an extracellular matrix bioscaffold assembled in vivo.

In a further embodiment, a method is provided for measuring the efficacy of one or more chemotherapeutic compounds against tumor cells taken from a subject diagnosed with cancer. More specifically, a method is provided in which tumor cells taken from the subject are grown within the extracellular matrix bioscaffold of the instant invention until one or more tumors are detected that are suitable for testing the compounds; a chemotherapeutic compound is administered to the extracellular matrix and the size of the tumor(s) is measured after sufficient time has elapsed for the compound to shrink the tumors. The tumor cells may be labeled with at least one biological reporter gene which is encoded with one or more expression vectors that are expressed within the tumor cell. Such biological reporter genes may encode at least one reporter selected from green fluorescence protein, luciferase, and combinations thereof.

Again, in a broader sense, methods are provided that measure the efficacy of tumor treatment methods against tumor cells taken from a subject diagnosed with cancer. In accordance with this embodiment, tumor cells taken from the subject are grown within the extracellular matrix bioscaffold of the present invention until a cellular matrix is obtained with tumors that are suitable for testing; the treatment method is then performed on the extracellular matrix; and the size of the tumors is measured after sufficient time has elapsed for the treatment method to shrink the tumors.

Further methods of the instant invention include adaptations on the foregoing for assaying the efficacy of a multi-dose course of treatment of one or more chemotherapeutic compounds or treatment methods against a tumor cell line. This method includes administering at fixed or variable time intervals over a course of treatment one or more chemotherapeutic compounds or therapeutic methods to the extracellular matrix bioscaffold of the instant invention, which includes tumors grown from the tumor cell line. The size of the tumors are then measured after sufficient time has elapsed from each administration for the compound to shrink the tumors. The fixed or variable intervals of administration may be specifically adapted to imitate a course of chemotherapeutic treatment in vivo for the given chemotherapeutic compound or therapeutic method. Alternatively, in embodiments where multiple chemotherapeutic compounds or methods are used, each sequentially administered at a given interval over the course of treatment.

The instant invention also relates to kits that may be used for performing such methods and/or assays. In one embodiment, extracellular matrix bioscaffold kits are provided for supporting the formation and growth of tumors from tumor cells introduced thereto that include either a container of carcinoma-associated fibroblast-like cells, a container of tumor associated macrophages, or both, and instructions for culturing the carcinoma-associated fibroblast-like cells and tumor associated macrophages under conditions effective to provide a cellular matrix capable of supporting the formation and growth of tumors from tumor cells introduced to the matrix and for incubating tumor cells in the matrix to support tumor growth. Kits according to this embodiment optionally include a container of tumor cells for incubation within the matrix. In further embodiments, this kit may be similarly used for measuring the efficacy of one or more chemotherapeutic compounds against tumor cells. Accordingly, the kit further includes instructions for measuring the efficacy of one or more chemotherapeutic compounds against the tumors.

In an alternative embodiment, kits for supporting the formation and growth of tumors from tumor cells on an extracellular matrix bioscaffold contain an extracellular matrix of precultured tumor associated macrophages and carcinoma-associated fibroblast-like cells. In another alternative embodiment, the precultured matrix contains either tumors already grown within the matrix or a container of tumor cells to be added to and grown within the matrix. The kit further includes instructions for culturing tumor cells to result in the growth and formation of tumors. In further embodiments, this kit may be used for measuring the efficacy of one or more chemotherapeutic compounds against tumor cells on an extracellular matrix bioscaffold. Accordingly, kits according to the present invention may further include instructions for measuring the efficacy of one or more chemotherapeutic compounds against the tumors.

### BRIEF DESCRIPTION OF THE FIGURES

FIG. 1 illustrates increased expression of myofibroblast marker proteins α-SMA, FSP, and vimentin in differentiated MSCs, as observed by immunofluorescence staining;

FIG. 2 illustrates observations of increased luciferase activity indicating that the three cell mixture supports growth of tumor cells in vitro;

FIG. 3 illustrates tumor size relative to the incubation period in a three cell mixture supporting growth of tumor cells in vitro;

FIG. 4 illustrates MDAMB231 tumors growing in nude mice with the left panel illustrating cell growth on BD Matrigel™ and the right panel illustrating growth of MDA, CAFs, and TAMs after 35 day incubation;

FIG. 5 illustrates MDAMB231 tumors growing in nude mice with the right panel illustrating cell growth on BD Matrigel™ and the left panel illustrating growth of MDA, CAFs, and TAMs after 25 day incubation;

FIG. 6 illustrates Jak2/Stat3 mediates SDF-1 signaling in MSCs;

FIG. 7A illustrates IL-8 mediated increase in SDF-1 mRNA expression by MSCs is decreased after PKC inhibition and FIG. 7B shows MSC migration is impaired by PKC zeta knockdown where trans-well migration assay was performed using MSCs after transfection;

FIG. 8 illustrates a proposed model for MSC signaling in the tumor microenvironment;

FIG. 9 illustrates inhibition of Jak/Stat3 signaling in tumor cells following dehydrodidemnin B treatment;

FIG. 10 illustrates growth of tumor cells in the reconstituted tumor microenvironment can be inhibited by dehydrodidemnin B;

FIG. 11 illustrates fibroblast surface protein (FSP) as a marker for tumor stroma; and

### DETAILED DESCRIPTION OF THE INVENTION

The present invention provides tumor microenvironments within which tumor cells may be grown. Microenvironments according to the present invention are created within an extra-cellular matrix bioscaffold of carcinoma-associated fibroblast-like cells and tumor associated macrophages. The microenvironment may be used for the testing, identification and development of known or novel anticancer therapeutics. As shown herein, the incubation of carcinoma-associated fibroblast-like cells and tumor associated macrophages with tumor cells led to tumor cell proliferation within in vitro cell cultures. Xenograft transplantation of the extracellular matrix bioscaffold cells of the present invention into laboratory test animals similarly led to the establishment in vivo of a tumor microenvironment promoting tumor cell growth.

Accordingly, the instant invention presents a novel extracellular matrix that mimics the native tumor milieu and achieves tumor cell growth and tumor formation in artificially induced in vitro and in vivo environments. Such a matrix is advantageously used in screening assays for known and novel therapeutics. The matrix is also adaptable for the isolation, culturing and drug testing of a patient-specific carcinoma and the development of a personalized, therapeutic approach for treating the patient. Additional advantages will be apparent to one of ordinary skill in the art based on the disclosure and examples provided herein.

The carcinoma-associated fibroblast-like cells of the instant invention refer to cell types exhibiting carcinoma-associated fibroblast (CAF) activity, particularly cell types leading to neoplastic progression, angiogenesis and/or metastasis. In one embodiment, carcinoma-associated fibroblast-like cells express the chemokine stromal-derived factor-1 (SDF-1). In further embodiments, the carcinoma-associated fibroblast-like cells are positive for one or more biological markers associated with CAFs. Such markers include, but are not limited to, α-smooth muscle actin (a-SMA), vimentin, and fibroblast surface protein.

The carcinoma-associated fibroblast-like cells of the instant invention may be derived or differentiated from pluripotent human bone marrow-derived cells. Such cells may include, for example, mesenchymal stromal cells (MSCs) that are differentiated to exhibit one or more of the foregoing phenotypes. One method for achieving MSC differentiation is by culturing the cell in tumor conditioned medium. More specifically, MSCs may be plated in tumor conditioned medium for about 1 to 30 days, with fresh medium being supplied regularly every several days. In certain embodiments, MSCs are plated onto tumor conditioned medium and incubated for 30 days, with fresh medium being supplied every third to fourth day. To this end, methods of differentiating MSCs may be in accordance with the methodology provided below. Alternatively, MSC isolation, culturing, and/or differentiated techniques may be in accordance with the methods disclosed in Mishra, P. J. et al “Carcinoma-Associated Fibroblast-Like Differentiation of Human Mesenchymal Stem Cells,” Cancer Res (2008); 68(11):4331-4339, the contents of which are incorporated herein by reference.

As used herein, “tumor-conditioned medium” is defined as a composition or medium, such as a culture medium, which contains one or more tumor-derived cytokines, lymphokines or other effector molecules. Most typically, tumor-conditioned medium is prepared from a culture medium in which selected tumor cells have been grown, and will therefore be enriched in such tumor-derived products. The type of medium is not believed to be particularly important, so long as it at least initially contains appropriate nutrients and conditions to support tumor cell growth. It is also possible to extract and separate materials from tumor-conditioned media and employ one or more of the extracted products for application to MSCs. In one embodiment, the tumor conditioned medium may be harvested Dulbecco's Modified Eagle's Medium (DMEM)+10% heat-inactivated FBS conditioned from the growth of one or more tumor cell types to be grown in the resulting microenvironment for 16 hours. In other embodiments, cells may be differentiated in the presence of the chemokine Interleukin-8 (CXCL8). Such tumor cells types may include, but are not limited to, one or a combination of breast cancer cells (e.g. MDAMB231 cells) and glioma cells (e.g. U87 cells) and/or pancreatic cancer cells (e.g. PANC1 cells).

Tumor associated macrophages of the instant invention refer to a population of leukocytes exhibiting a macrophage phenotype that promotes tumor cell proliferation, metastasis and/or angiogenesis, or otherwise promotes chemotaxis of MSCs. Such cells may be derived from a leukemic or lymphoma cell line that is differentiated to exhibit one or more of the foregoing phenotypes. For example, in one non-limiting embodiment, the tumor associated macrophages of the instant invention are derived from human promyelocytic leukemia cells, such as but not limited to HL-60. Alternatively, the tumor associated macrophages may be derived from a histiocytic lymphoma, such as but not limited to U937.

Differentiation of leukocytes into tumor associated macrophages may be accomplished by culturing the cells in the presence of 1-20 nM of a tumor promoter for 1 to 96 hours using one or more methodologies discussed herein or otherwise know in the art. Tumor promoters that may be used to such purposes include phorbol esters. In a non-limiting embodiment, the phorbol ester is 12-O-tetradecanoylphorbol-13-acetate (TPA).

In one non-limiting embodiment differentiation of a leukemic or lymphoma cell line into tumor associated macrophages is accomplished by incubating such cells in the presence of 3 nM of TPA for 96 hours. In a further non-limiting embodiment 1.5×10⁶ of HL-60 cells are plated on RPMI media in tissue culture flask and differentiated for 4 days in the presence of 3 nM PMA, with the media being changed every 3-4 days. Methods of leukocyte differentiation also may be in accordance with the methodology provided below, or otherwise as set forth in Rovera, G. et al. “Human Promyelocytic leukemia cells in culture differentiate into macrophage-like cells when treated with a phorbol diester” PNAS (1979) 76(6):2779-83; or Fontana, J. A. et al “Identification of a population of bipotent stem cells in the HL60 human promyelocytic leukemia cell line,” PNAS (1981) 78(6):3863-66, the contents of which are incorporated herein by reference.

As used herein, the terms “tumor cells,” “cancer cells,” and “carcinomas” are inclusive of all such cell types known in the art, including but not limited to fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor cells, leiomyosarcoma, rhabdomyosarcoma, colon carcinoma, pancreatic cancer cells, breast cancer cells, ovarian cancer cells, prostate cancer cells, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor cells, cervical cancer cells, testicular tumor cells, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstrobm's macroglobulinemia, and heavy chain disease. In certain embodiments, the instant invention is related to epithelial cell carcinomas. Such cells may be provided from one or more cell lines that are known in the art. Alternatively, as discussed in greater detail below, such cell types may also be isolated directly from a subject or patient.

In certain embodiments, the tumor cell is transfected with or otherwise labeled with one or more biological reporter genes, which are used to measure or detect tumor cell growth in the microenvironment. In one embodiment, the biological reporter genes are one or a combination of green fluorescence protein (GFP) and luciferase genes (Luc IRES GFP). Such genes are inserted within a known expression vector, downstream of a constitutively active promoter and transfected into the tumor cells using standard methods known in the art. Such methods may include variations of those disclosed within Sambrook et al. (2001) Molecular Cloning: A Laboratory Manual (3d ed., Cold Spring Harbor Laboratory Press, Plainview, N.Y.), the contents of which are incorporate herein by reference.

The instant invention is not necessarily limited to one or a combination of the foregoing reporter genes and may include reporter genes otherwise known in the art, such as, but not limited to, enhanced GFP (EGFP), β-galactosidase, alkaline phosphatase, and/or chloramphenicol acetyl transferase.

Reconstitution of the tumor microenvironment including these three cell types may be provided either in vitro or in vivo as a xenograft transplant. Extracellular matrix bioscaffolding products for both types of testing are commercially available and well-known. The products are marketed for culturing tumors for the investigation of therapeutic drugs and treatment methods. Techniques for culturing tumors in the extracellular matrix products of the present invention are similar to those employed with existing commercial products. However, the incorporation of carcinoma-associated fibroblast-like cells and tumor-associated microphages into the extra-cellular matrices of the present invention better approximates conditions present in tumor micro-environments, thereby providing a more accurate measure of how candidate drug compounds and therapies will perform in human clinical trials.

With respect to in vitro reconstitution, this is provided by incubating the tumor cells, carcinoma-associated fibroblast-like cells, and tumor associated macrophages on cell growth medium using one or a combination of the protocols provided herein, which may include elements of protocols used with existing products adapted to the used of the fibroblast-like cells and microphages. In one non-limiting embodiment, the tumor associated macrophages may be first established on a cell growth medium by differentiating a leukemic or lymphoma cell line in accordance with the teachings herein. Carcinoma-associated fibroblast-like cells are then added to the medium and co-cultured with the tumor associated macrophages, establishing an extra-cellular matrix bioscaffold that is capable of supporting the formation and growth of tumors from tumor cells introduced thereto. After a brief incubation period tumor cells are then added to the culture and incubated at 37° C. and 5% CO₂. Although not limited thereto, the ratio of tumor associated macrophages to carcinoma-associated fibroblast-like cells to tumor cells is between 1:1:1 to 1:1:5. Tumor growth is then confirmed using one or more of the biological reporters provided herein and associated methods known in the art. One of ordinary skill in the art will appreciate that the instant protocol is not necessarily limiting to the invention and may be varied in accordance with the cells types used and the teachings herein.

Reconstitution of the tumor microenvironment in vivo may be provided by incubating the tumor cells, carcinoma-associated fibroblast-like cells, and tumor associated macrophages within a test subject. In one non-limiting embodiment, tumor cells, the tumor associated macrophages and carcinoma-associated fibroblast-like cells are injected into NCR nude mice and incubated in accordance with the teachings herein. Again, the carcinoma-associated fibroblast-like cells and tumor associated macrophages provide for the extracellular matrix bioscaffold that supports growth of the tumor cells and, ultimately, formation of tumors. Although not limited thereto, the ratio of tumor associated macrophages:carcinoma-associated fibroblast-like cells:tumor cells is between 1:1:1 to 1:1:5. Using the biological reporter, and associated methods, tumor formation is tracked until reaching a desired size, or a size suitable for testing one or more therapeutic agents. In one embodiment, the tumor formation is tracked until the tumor reaches at least 1.5 cm in diameter.

The reconstituted tumor microenvironments of the instant invention may be used to study the impact of the tumor milieu on tumor growth and response to therapy such as the efficacy of a chemotherapeutic compound or other therapeutic strategy against a tumor cell line. More specifically, the instant environment creates conditions that mimic the true in vivo conditions observed in the native environment of the tumor cells. One or more novel or otherwise known therapeutics can then be administered to the cells. The influence of the therapeutic on the size of the tumor cells, i.e., the growth or shrinking of the tumor, is then monitored by the biological reporter in both an in vitro as well as in vivo conditions, as provided above. To this end, one of ordinary skill in the art that the size of the tumors grown in the matrix are measured before administration of the therapeutic to establish a baseline. The therapeutic is then administered and its effects on the tumor measured a sufficient time after administration. A sufficient time after administration may include any amount of time necessary for the agent or therapeutic to affect the targeted cells or pathway. It may include, but is not limited to, 1 hour, 2 hours, 12 hours, 24 hours, 48 hours, or any other time period consistent with the teachings herein.

In another embodiment, the reconstituted tumor microenvironment may be utilized to establish novel targets for drug therapeutics. For example, targeting individual components of the microenvironment, rather than tumor cell mechanism, could lead to disruption of the tumor-stroma dialog and impair growth of tumors. Accordingly, the instant reconstituted tumor micro-environment would be used to further study tumor-stroma interaction, and test novel chemotherapeutic agents against such targets.

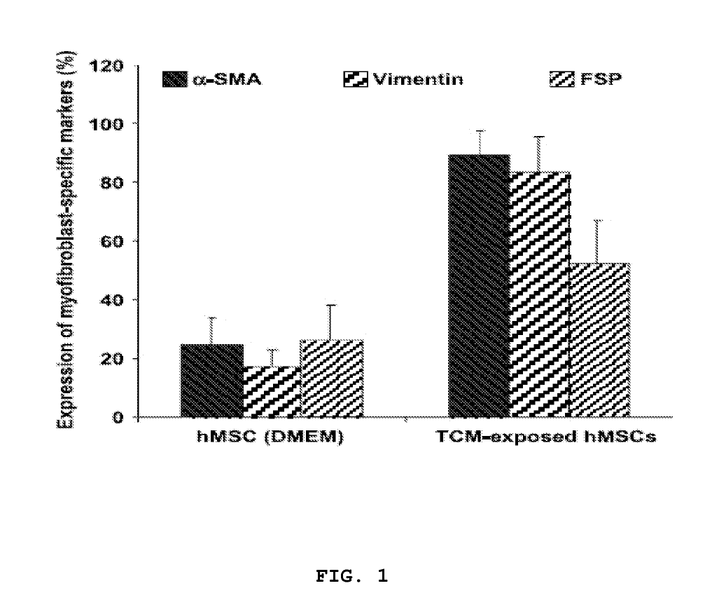

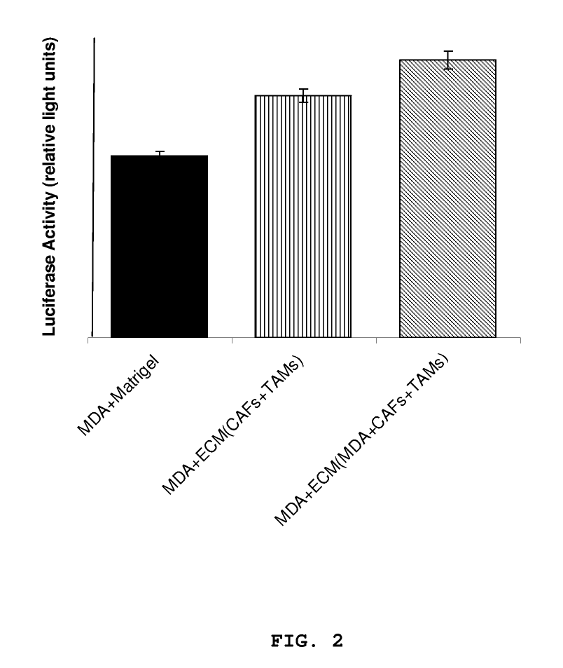

In a further non-limiting embodiment, such a novel target could be a pathway associated with the expression of SDF-1. The involvement of SDF-1 in growth of primary tumors and cell recruitment in the tumor microenvironment has been demonstrated. SDF-1 downstream signaling, for example, is believe to be mediated via STAT3 and ERK/MAPK pathways (FIG. 1). Additionally, tumor-secreted factor, IL-8, stimulates phosphorylation and activation of specific isoforms of PKC (PKC zeta) leading to SDF-1 expression (FIG. 2). Because of their role in activation of CAFs in tumor microenvironment, these pathways present possible targets for chemotherapeutic intervention.

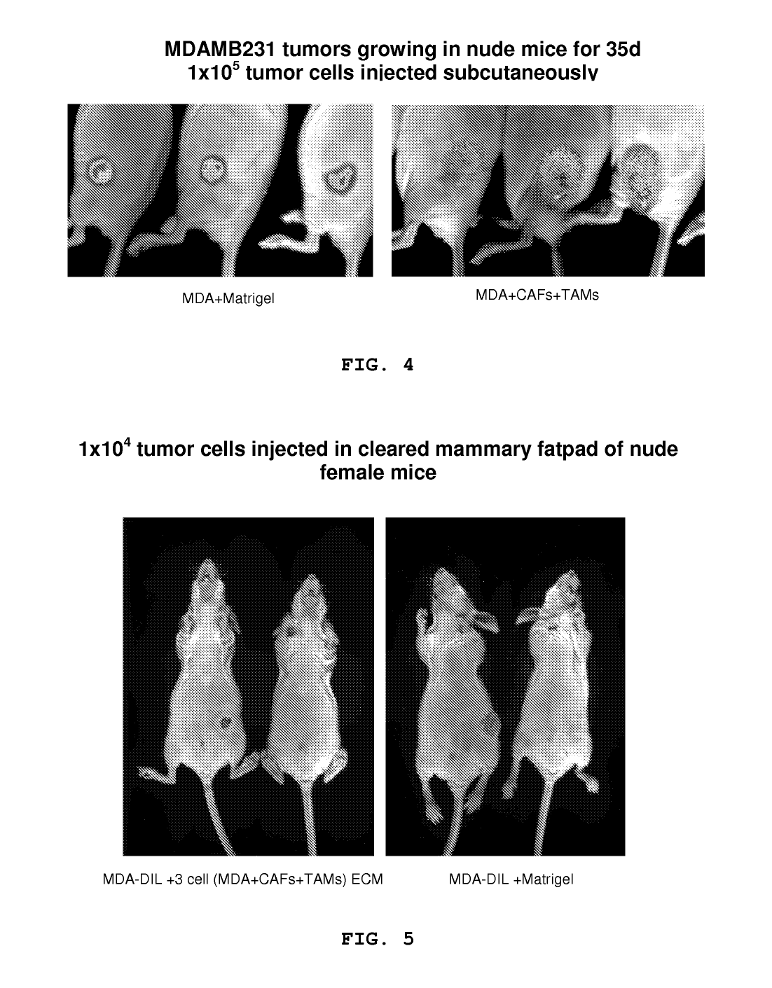

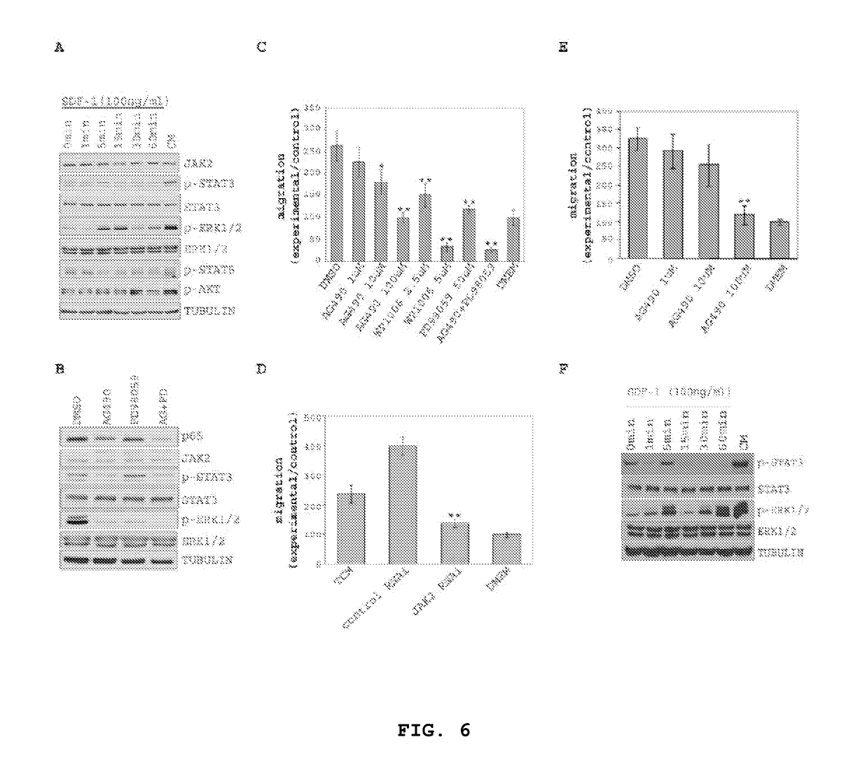

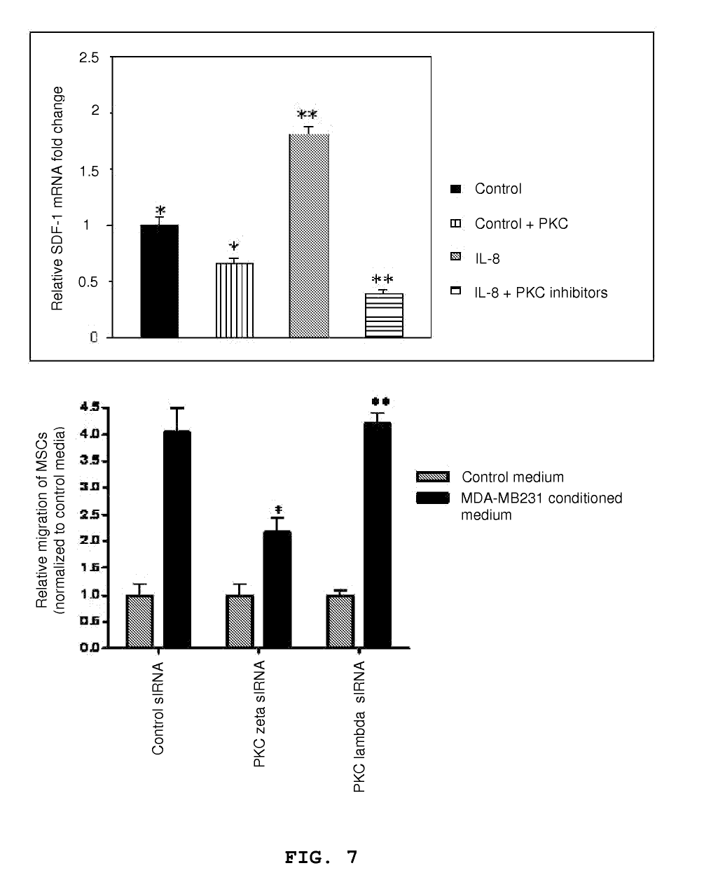

As illustrated in FIG. 4, inhibition of Jak/Stat3 signaling following dehydrodidemnin B (plitidepsin) treatment led to the demise of tumor cells. Tumor cell growth was similarly inhibited by administering dehydrodidemnin B to tumor cells established within the reconstituted tumor microenvironment of the instant invention (FIG. 5). In addition dehydrodidemnin B treatment also resulted in decreased FSP positive tumor stroma (FIG. 6). Accordingly, the instant invention is useful in drug screening assays for known and novel therapeutics targeting either the tumor cell or components of the tumor cell environment.

Apart from use in drug screening assays, the reconstituted tumor microenvironment also may be used to establish a patient-specific therapeutic regime, based on the particular carcinoma genotype and/or phenotype. One or more tumor cells are isolated from the patient and equipped with biological reporters in accordance with the teachings herein. The extracellular matrix bioscaffold may then be provided, as described herein, and the isolated carcinoma cells grown into tumors thereon. The isolated carcinoma are then tested against one or more therapeutics. To this end, a patient specific cell line is established and a therapeutic regime tested prior to administration to the patient.

The foregoing embodiments for testing the therapeutic effects of one or more agents on a tumor cell line or resulting tumors may be further adapted to mimic or otherwise establish an optimum course of therapy envisioned for a patient. To this end, one or more therapeutic agents may be administered to tumors grown on the extracellular matrix bioscaffold at fixed or variable time intervals over a course of treatment. In embodiments wherein multiple agents are used, the agents may be administered successively at each interval or otherwise using protocols known in the art for administering such compounds. As used herein a “fixed interval” refers to a fixed time course or fixed rate for regularly administering a dosage form. A “variable time” course refers to the administration of dosages at variable intervals during a time course, such as an “as needed” basis. One of ordinary skill in the art will appreciate that the course of treatment may be established based on the desired or optimal cumulative dosage to be administered to the patient for purposes of completing the treatment or otherwise eradicating the tumor and tumor cells. In one embodiment, a course of treatment and intervals may be established based on the response of the tumor cells and tumor shrinkage in the extracellular matrix bioscaffold of the instant invention.

Further embodiments of the instant invention include one or more kits for supporting the formation and growth of tumors from tumor cells introduced thereto and for testing the efficacy of one or more therapeutic agents. In one embodiment the kit includes either carcinoma-associated fibroblast-like cells, tumor associated macrophages, or both, and a set of instructions for culturing carcinoma-associated fibroblast-like cells and tumor associated macrophages to provide the extracellular matrix bioscaffold and for incubating tumor cells in the matrix to support tumor growth, as provided herein. The instructions may also include the steps required for measuring the efficacy of one or more chemotherapeutic compounds against the tumors, as provided herein.

In an alternative embodiments, the kit includes a pre-cultured extracellular matrix bioscaffold of the instant invention and instructions for culturing tumor cells introduced to the matrix to result in the growth and formation of tumors, as provided herein. The instructions may also include the steps for measuring the efficacy of one or more chemotherapeutic compounds against the tumors, as provided herein.

In another alternative embodiment, the kit includes a pre-cultured extracellular matrix bioscaffold with tumors already grown on the extracellular matrix bioscaffold from a tumor cell line and instructions for measuring the efficacy of one or more chemotherapeutic compounds against the tumors.

Each of the foregoing kits may be adapted in accordance with one or more of the teachings herein.

The following non-limiting examples set forth hereinbelow illustrate certain aspects of the invention.

### EXAMPLES

### Materials and Methods

### Tumor Cells

Breast cancer cell line, MDAMB231 (American Type Culture Collection) were cultured in DMEM (Life Technologies) supplemented with 10% FBS and penicillin-streptomycin at 37° C. in 5% CO₂.

### Biological Reporter

Breast cancer cell line MDAMB231 was transfected with a mammalian expression vector bearing both green fluorescence protein (GFP) and luciferase genes (Luc IRES GFP) under a CMV promoter and stable GFP and luciferase-expressing clones are selected using a variation of the methods disclosed in Sambrook et al. (2001) Molecular Cloning: A Laboratory Manual (3d ed., Cold Spring Harbor Laboratory Press, Plainview, N.Y.), the contents of which are incorporate herein by reference.

### Isolation and Culture of Mesenchymal Stem Cells

Human MSCs were obtained from bone marrow samples either purchased from commercial sources (Lonza, MD) or from samples collected from healthy volunteers who were compensated for their donation as per IRB guidelines and an IRB approved protocol. The cells were then plated in T150 or T 75 cm² flasks with Minimum Essential Medium alpha medium (α MEM) containing 10% fetal bovine serum (FBS) and penicillin/streptomycin. The cultures were incubated at 37° C. in a humidified atmosphere containing 5% CO₂. Nonadherent cells were removed after 24 h, and the medium was changed every other day. Adherent cells were detached from the flasks by treatment with 0.05% trypsin and ethylenediaminetetraacetic acid (EDTA) and subcultured every 4 to 5 days and aliquots from passage 3 to 5 are frozen in liquid nitrogen for future use. Early passage cells were tested for their capacity to differentiate in culture.

### Activated MSCs

Tumor conditioned medium (TCM) was harvested from cultures of MDAMB231 cells and glioma (U87) and pancreatic cancer (PANC1). More specifically, MDAMB231, U87, and PANC1 cells were grown in RPMI+10% heat inactivated FBS (heat inactivation is carried out at 56° C. for 30 min) culture medium. The tumor conditioned medium was harvested 12-14 h following change of culture medium for cells in logarithmic growth phase. MSCs were then cultured in the presence of the TCM and analyzed for carcinoma-associated fibroblast-like differentiation.

MSCs were exposed to TCM for 30 days with fresh TCM added every 4 days as described in Mishra et al., “Carcinoma-Associated Fibroblast-like Differentiation of Human Mesenchymal Stem Cells,” Cancer Res (2008) 68(11): 4331-9, the contents of which are incorporated herein by reference.

Referring to FIG. 1, carcinoma-associated fibroblast-like cells were characterized by increased expression of α-smooth muscle actin, vimentin and fibroblast surface protein among others. Naïve MSCs expressed little or no α-smooth muscle actin, vimentin or fibroblast surface protein while activated human MSCs expressed increased amounts of a-smooth muscle actin, vimentin and fibroblast surface protein indicating that the TCM exposed MSCs were differentiating into myofibroblast-like cells. FIG. 1 shows results of quantitation of immunofluorescence. All samples were counterstained with DAPI to visualize nuclei and appear blue in photographs. Results shown are for MDAMB231CM exposed MSCs.

To further measure differentiation, primers for SDF-1 and 18s rRNA were designed using software available from ABI Biosystems and obtained from Dharmacon (Boulder, Colo.). SDF-1 mRNA levels are determined for each MSCs condition in four independent experiments and each quantitative RT-PCR is carried out in quadruplicate. Preparation of RNA and reverse transcription is carried out using commercially available kits from Invitrogen (Carlsbad, Calif.). Tubes are incubated at 50° C. for 30 min for reverse transcription followed by a denaturation step at 94° C. for 2 min. This is followed by 25 cycles of PCR amplification at 94° C. for 15 sec; 55° C. for 30 sec and 72° C. for 1 min. The final elongation step is carried out at 72° C. for 7 min. SDF-1 sense primer sequence: 5′-TTTGAGAGCCATGTCGCCA-3′ (SEQ ID NO: 1) antisense primer sequence: 5′-TGTCTGTTGTTGCTTTTCAGCC-3′ (SEQ ID NO: 2). Eukaryotic 18S rRNA (TaqMan pre-Developed Assay Reagent) was used as endogenous control. Levels of SDF-1 expression were reported as a ratio of SDF-1 to 18sRNA and were considered to be 100 percent for the naïve MSCs. Changes in levels of SDF-1 mRNA were reported as percent changes from naïve MSC levels.

Alternatively, MSCs were differentiated by exposure to 10 ng/ml CXCL8.

### Tumor Associated Macrophages

The human leukemia cell line HL-60 was used as a macrophage model in these studies. HL-60 cells were differentiated to a macrophage phenotype using 3 nM PMA according to the protocol disclosed in Rovera, G. et al. “Human Promyelocytic leukemia cells in culture differentiate into macrophage-like cells when treated with a phorbol diester” PNAS (1979) 76(6):2779-83, the contents of which are incorporated herein by reference. More specifically, 1.5×10⁶ HL-60 cells were cultured in RPMI media in tissue culture flask and differentiated for 4 days into TAMs (Tumor associated macrophages) by 3 nM PMA, changed media regularly. Differentiation was confirmed by a differentiated tumor macrophage phenotype that promotes MSC chemotaxis; undifferentiated HL-60 cells do not stimulate MSC chemotaxis.

### Example 1

### Three Cell Culture & Differentiation

1.5×10⁶ HL-60 cells were cultured in RPMI media in tissue culture flask and differentiated for 4 days into TAMs (Tumor associated macrophages) by 3 nM PMA, changed media regularly. After day four, 5×10⁶ carcinoma associated fibroblast-like cells were added & co-cultured 2 additional days. After the second day, 2×10⁶ MDAMB231 cells were added and cultured for 3 additional days. Simultaneously another culture was established with MDAMB231 as a two cell control. Also, MDAMB231 cells were cultured in Matrigel™ (BD Biosciences). After the third day cells, were scraped and collected in a 15 ml tube and centrifuged at 900 rpm for 5 mins. At this point pellet was stored and frozen in −80° C. for ECM isolation.

Referring to FIG. 2, MDAMB231 cells (luciferase expressing) were used to assay the effect of reconstituted tumor microenvironment on breast cancer cell growth. Luciferase activity was measured using D-luciferin as substrate in a luminometer. Y axis shows relative light units. Data shows dependence of MDAMB231 cells (representing basal subtype) on reconstituted tumor milieu for growth.

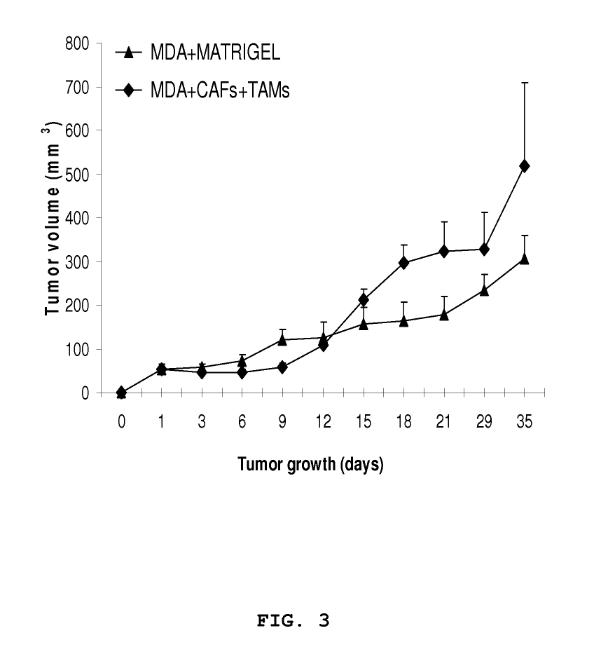

MDAMB231 cells were similarly incubated in the presence of both TAMs and carcinoma associated fibroblast-like cells for 40 days. The growth rate of the tumors in the microenvironment were compared with tumor cell growth in a commercially available reconstituted basement membrane, Matrigel™. As illustrated in FIG. 3, tumor growth in the presence of TAMs and CAF-like cells exceeded that of the Matrigel™.

### Example 2

### Three Cell In-Vivo Tumor Formation

Xenograft studies in athymic mice are carried out according to guidelines outlined in an animal use protocol approved by the IACUC. Noninvasive bioluminescence imaging monitors tumor growth.

TAMs and carcinoma associated fibroblast-like cells were injected S.C. along with MDAMB231 breast cancer cells in ratios between 1:1:1 to 1:1:5 in NCR nude mice and followed for tumor formation for 5-6 weeks. The tumor was dissected under sterile condition after reaching 1.5 cm diameter size and stored at −80° C. for ECM isolation.

Referring to FIGS. 4, 5 and 3, carcinoma-associated fibroblast-like cells and TAMs when injected together with MDAMB231 cells result in robust tumor growth in nude mice (n=5 for all groups). MDAMB231 cells along with or without TCM-exposed hMSCs, TAMs, or Matrigel™ were injected and palpable tumors were seen on day 8 (tumor cells injected on day 0). The human breast cancer cells MDAMB231 when injected alone did not form tumors in nude mice. Matrigel as well as CAFs and TAMs together increase growth of MDAMB231 tumors in nude mice.

### Example 3

### ECM Isolation

Isolation was performed based on a previously described protocol. Briefly, frozen 3 cell pellet/3 cell derived frozen tumor, were pulverized using a mortar pestle in liquid LN2 and extracted in a high-salt/N-ethylmaleimide solution (3.4 mol/L NaCl, 50 mmol/L Tris-HCl, pH 7.4, 4 mmol/L ethylenediaminetetraacetic acid, 2 mmol/L N-ethylmaleimide) containing proteinase inhibitor cocktail at 4° C. Homogenates were enriched for ECM by two cycles of centrifugation (RCFₘₐₓ 110,000×g, 30 mins, 4° C.), and pellets resuspended in high-salt/N-ethylmaleimide buffer. ECM-enriched pellets were resuspended in mid-salt/urea solution (2 mol/L urea, 0.2 mol/L NaCl, 50 mmol/L Tris-HCl, pH 7.4, 4 mmol/L ethylenediaminetetraacetic acid, 2 mmol/L N-ethylmaleimide) with proteinase inhibitor cocktail and extracted overnight at 4° C. Samples were pelleted at RCFₘₐₓ 110,000×g, and ECM-enriched supernatants extensively dialyzed (MWCO 12-14,000 kd) against low-salt buffer (0.15 mol/L NaCl, 50 mmol/L Tris-HCl, pH 7.4, 4 mmol/L ethylenediaminetetraacetic acid), followed by dialysis against serum-free media [Dulbecco's modified Eagle's medium (Gibco) supplemented with 1 μg/ml gentamicin] at 4° C. Matrix protein extract was stored at −80° C.

### Example 4

### Drug Sensitivity Assays in Reconstituted System

The three cell system in Examples 1 and 2 is prepared and used to test standard chemotherapeutic agents for activity against cancer cell lines, such as the breast cancer cell lines MDAMB231 and MDAMB435 representing basal subtype and MCF7 and SKBR3 representing luminal subtype of breast cancer. Reconstitution is carried out in 96 well plates by co-culturing a fixed number of tumor cells and carcinoma-associated fibroblast-like cells plus TAMs either alone or admixed with differing amounts of cells representing cell types in tumor milieu. Tumor growth is monitored at these sites in accordance with the foregoing. As the tumor cells express luciferase, tumor growth is monitored even at early time points by bioluminescence imaging following injection of D-luciferin on a Kodak MM2000 Imager. Again, all the tumor cell lines tested in culture are tested in this assay. These data can be used to create growth curves.

The objective is determination of tumor milieu influences on the sensitivity of tumor cells to chemotherapeutic agents. Moreover, these studies are also carried out under hypoxia to determine if sensitivity to drugs is altered under hypoxic conditions. This system is used widely for determining sensitivity of tumor cells to both old and newly developed drugs. Given that the tumor microenvironment more accurately represents the native environment of the cells, this system more accurately reflects in vivo drug sensitivity and allows investigators to screen drugs in a meaningful manner thereby reducing animal numbers for these kinds of preclinical assays

### Example 5

### Determine Effect of Reconstituted Tumor Milieu on Tumor Cell Growth In Vivo

The impact of activated MSCs on tumor cell growth in xenograft models in female nude mice (nu/nu or nude beige) is also measured. The breast cancer cell line MDAMB231 bearing luciferase transgene is again utilized in these experiments. Specifically, a fixed number of these luc positive cells, either alone or admixed with differing amounts of activated MSCs representing CAFs are injected into flanks of nude mice and tumor growth monitored by bioluminescence imaging following injection of D-luciferin (150 mg/kg body weight). Serial imaging on the Kodak MM2000 Imaging Station (Carestream Molecular Imaging, New Haven, Conn.) is carried out to monitor tumor growth (admixed with activated MSCs). Quantitation of signals from imaging is carried out using KODAK software using “region of interest” intensity and tumor volumes determined by plotting on standard curve (established earlier with MDAMB231 cells admixed with varying amounts of activated MSCs). Tumor volumes are calculated from signal intensity from “region of interest” (ROI) over the entire experimental period. These data is used to create growth curves. As illustrated in FIGS. 4 and 5, preliminary data with nu/nu as well as nude beige mice show good correlation between tumor volume and bioluminescence. The effect of different admixed cells on xenograft growth curves is clearly measured by this technique, and will be more sensitive than bulk tumor measurements.

### Example 6

### Use of the Tumor Microenvironment to Study Tumor-Stroma Dialog

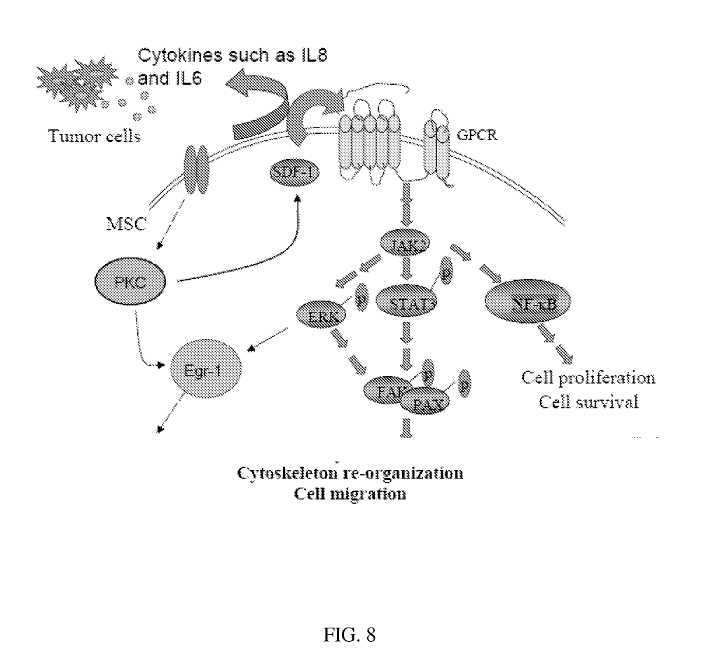

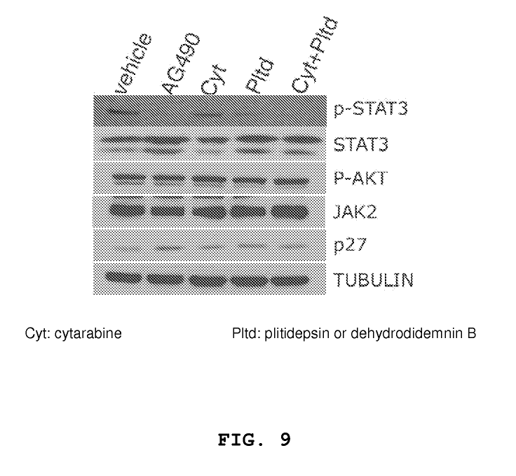

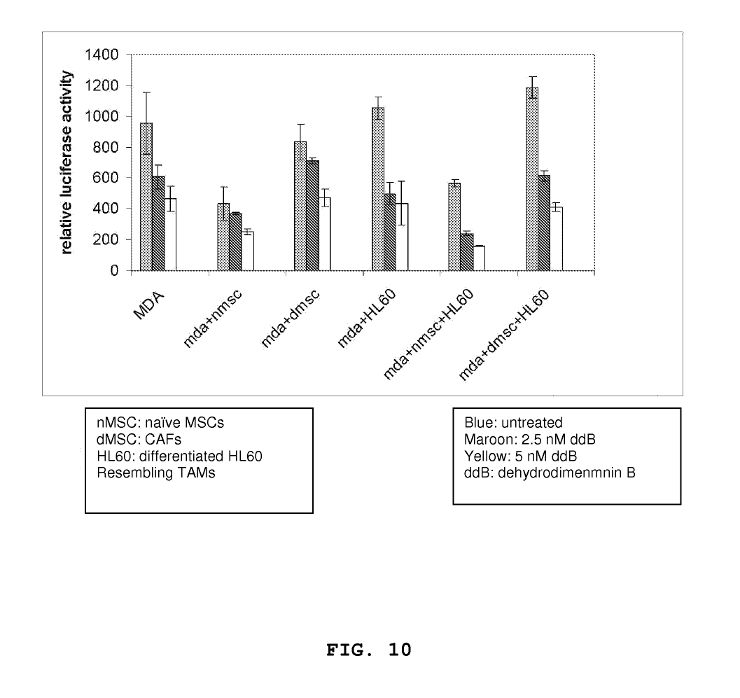

SDF-1 downstream signaling was identified to be mediated via STAT3 and ERK/MAPK pathways (FIG. 8). Moreover, tumor-secreted factor, IL-8, stimulated phosphorylation and activation of specific isoforms of PKC (PKC zeta) leading to SDF-1 expression (FIG. 9). Based on a foregoing, a proposed model for cell signaling in the tumor microenvironment is shown in FIG. 10. Because of their role in activation of CAFs in tumor microenvironment, these pathways may be key targets for chemotherapeutic intervention.

The reconstituted system is used to identify synergistic combinations using a panel of standard chemotherapeutic agents and Jak/STAT3 inhibitors. The Chou Talalay combination index analysis is used and the data analyzed using Calcusyn software (Biosoft ver 3). Once potential synergistic combinations from the in vitro combination index analysis are identified, the combination in vivo using breast cancer cells (luc expressing) in reconstituted tumor milieu system are tested. Growth of tumors is monitored by bioluminescence imaging using a KODAK 2000mM Imaging station as described earlier. This model is further extended to breast tumor growth in preclinical models in vivo to determine the effect of these agents in combination with standard chemotherapeutic agents in tumors with a highly desmoplastic stroma such as basal like breast cancers in pre-clinical models.

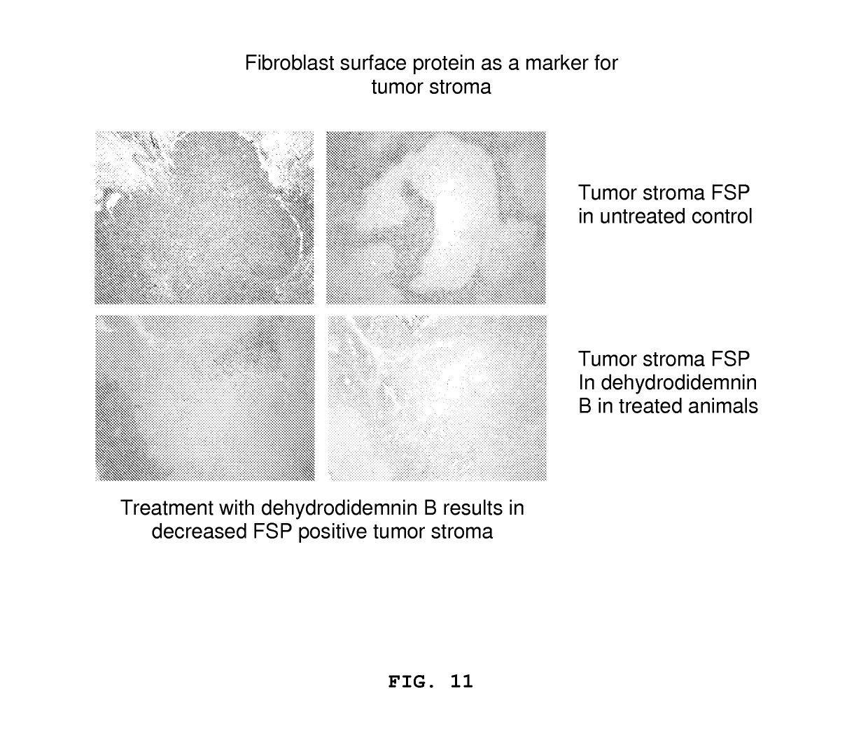

The instant system elicited the inhibition of Jak/Stat3 signaling in tumor cells following dehydrodidemnin B (plitidepsin) treatment (FIG. 9). To this end, the growth of tumor cells in the reconstituted tumor microenvironment was inhibited by Dehydrodidemnin B (FIG. 10). dehydrodidemnin B treatment also resulted in decreased FSP positive tumor stroma (FIG. 11).

The foregoing examples and description of the preferred embodiments should be taken as illustrating, rather than as limiting the present invention as defined by the claims. As will be readily appreciated, numerous variations and combinations of the features set forth above can be utilized without departing from the present invention as set forth in the claims. Such variations are not regarded as a departure from the spirit and script of the invention, and all such variations are intended to be included within the scope of the following claims.

1. An extracellular matrix bioscaffold capable of supporting the formation and growth of tumors from tumor cells introduced thereto, said matrix comprising tumor associated macro-phages and carcinoma-associated fibroblast-like cells that are cultured under conditions effective to provide a cellular matrix capable of supporting the formation and growth of tumors from tumor cells introduced to said matrix.

2. The extracellular matrix of claim 1, wherein said carcinoma-associated fibroblast-like cells are comprised of mesenchymal stem cells that are differentiated on a tumor conditioned medium.

3. The extracellular matrix of claim 1, further comprising tumor cells introduced to said matrix.

4. The extracellular matrix of claim 3, further comprising tumors grown from said tumor cells.

5. The extracellular matrix of claim 3, wherein said tumor cells comprise one or more biological reporter genes.

6. The extracellular matrix of claim 5, wherein at least one said biological reporter genes encode a reporter selected from the group consisting of green fluorescence protein, luciferase, and combinations thereof.

7. The extracellular matrix of claim 1, wherein the tumor associated macrophages promote chemotaxis of mesenchymal stem cells.

8. The extracellular matrix of claim 1, wherein the tumor associated macrophages are phorbol ester differentiated HL-60 cells or U937 cells.

9. The extracellular matrix of claim 1, wherein the carcinoma-associated fibroblast-like cells express stromal-derived factor-1.

10. The extracellular matrix of claim 1 wherein the carcinoma-associated fibroblast-like cells are positive for one or more biological markers selected from the group consisting of α-smooth muscle actin, vimentin, and fibroblast surface protein.

11. A method for assaying the efficacy of a chemotherapeutic compound against a tumor cell line, said method comprising:
administering said chemotherapeutic compound to an extracellular matrix bioscaffold according to claim 1 comprising tumors grown from said cell line; and
measuring the size of said tumors after sufficient time has elapsed for said compound to shrink said tumors.

12.-14. (canceled)

15. The assay method of claim 11 wherein tumor size is determined by comparing a first value derived from a biological reporter before administration of the compound with a second value derived from the biological reporter after administration of the compound.

16.-19. (canceled)

20. A method of providing a tumor microenvironment for testing one or more compounds that treat cancer comprising: incubating tumor cells in an extracellular matrix bioscaffold comprising tumor associated macrophages and carcinoma-associated fibroblast-like cells under conditions effective to support the growth of tumors from said tumor cells.

21. The method of claim 20 wherein the carcinoma-associated fibroblast-like cells are comprised of mesenchymal stem cells that are differentiated on tumor conditioned medium for about 1 to 30 days.

22.-25. (canceled)

26. The method claim 20 wherein the tumor associated macrophages are phorbol ester differentiated HL-60 cells or U937 cells that are differentiated in the presence of 3 nM of TPA for 96 hours.

27.-29. (canceled)

30. The method of claim 20 wherein said tumor cells are incubated between about one and about four days.

31. The method of claim 20 wherein said tumor cells are incubated in an extracellular matrix bioscaffold assembled in vitro.

32. The method of claim 20 wherein said tumor cells are assembled in an extracellular matrix bioscaffold assembled in vivo.

33.-40. (canceled)

41. A method for assaying the efficacy of a tumor treatment method against a tumor cell line, said method comprising:
performing said treatment method on an extracellular matrix bioscaffold according to claim 1 comprising tumors grown from said cell line; and
measuring the growth or shrinkage of said tumors after sufficient time has elapsed for said treatment method to shrink said tumors.

42. (canceled)

43. A method for assaying the efficacy of one or more chemotherapeutic compounds against a tumor cell line, said method comprising:
administering the one or more chemotherapeutic compounds to an extracellular matrix bioscaffold according to claim 1 further comprising tumors grown from said cell line wherein the chemotherapeutic compounds are administered at fixed or variable time intervals over a course of treatment; and
measuring the size of said tumors after sufficient time has elapsed from each administration for said compound to shrink said tumors.

44. The method of claim 43 wherein the fixed or variable intervals of administration imitate a course of chemotherapeutic treatment for a subject with the chemotherapeutic compound.

45. The method of claim 43 wherein multiple chemotherapeutic compounds are sequentially administered at a given interval over the course of treatment.

46. A method for assaying the efficacy of one or more therapeutic methods against a tumor cell line, said method comprising:
administering the one or more therapeutic methods to an extracellular matrix bioscaffold according to claim 1 further comprising tumors grown from said cell line wherein the therapeutic methods are administered at fixed or variable time intervals over a course of treatment; and
measuring the size of said tumors after sufficient time has elapsed from each administration for said compound to shrink said tumors.

47. A kit for establishing an extracellular matrix bioscaffold capable of supporting the formation and growth of tumors from tumor cells introduced thereto comprising:
carcinoma-associated fibroblast-like cells, or tumor associated macrophages, or both; and
instructions for culturing the carcinoma-associated fibroblast-like cells and tumor associated macrophages under conditions effective to provide a cellular matrix capable of supporting the formation and growth of tumors from tumor cells introduced to said matrix and for incubating tumor cells in said matrix to support tumor growth.

48. A kit for measuring the efficacy of one or more chemotherapeutic compounds against tumor cells comprising:
carcinoma-associated fibroblast-like cells, or tumor associated macrophages, or both; and
instructions for culturing the carcinoma-associated fibroblast-like cells and tumor associated macrophages under conditions effective to provide a cellular matrix capable of supporting the formation and growth of tumors from tumor cells introduced to said matrix, instructions for incubating tumor cells in said matrix until tumors are obtained that are suitable for testing said compounds, and instructions for measuring the efficacy of one or more chemotherapeutic compounds against said tumors.

49. A kit for supporting the formation and growth of tumors from tumor cells comprising:
an extracellular matrix bioscaffold comprising tumor associated macrophages and carcinoma-associated fibroblast-like cells that are cultured under conditions effective to provide a cellular matrix capable of supporting the formation and growth of tumors from tumor cells introduced to said matrix; and
instructions for culturing tumor cells introduced to said matrix to result in the growth and formation of tumors.

50. A kit for measuring the efficacy of one or more chemotherapeutic compounds against tumor cells comprising:
an extracellular matrix bioscaffold comprising tumor associated macrophages and carcinoma-associated fibroblast-like cells that are cultured under conditions effective to provide a cellular matrix capable of supporting the formation and growth of tumors from tumor cells introduced to said matrix; and
instructions for culturing tumor cells introduced to said matrix to result in the formation and growth of tumors suitable for testing said compounds, and instructions for measuring the efficacy of one or more chemotherapeutic compounds against said tumors.

51. A kit for measuring the efficacy of one or more chemotherapeutic compounds against tumor cells comprising:
an extracellular matrix bioscaffold and tumors grown on said extracellular matrix bioscaffold from a tumor cell line, wherein said extracellular matrix bioscaffold comprises tumor associated macrophages and carcinoma-associated fibroblast-like cells that are cultured under conditions effective to provide a cellular matrix capable of supporting the formation and growth of said tumors from said tumor cell line; and
instructions for measuring the efficacy of one or more chemotherapeutic compounds against said tumors.


SEQUENCE LISTING (2 sequences)

SEQ ID NO: 1 - DNA, 19 bp, Artificial
  SDF-1 sense primer
tttgagagccatgtcgcca

SEQ ID NO: 2 - DNA, 22 bp, Artificial
  SDF-1 antisense primer
tgtctgttgttgcttttcagcc